(19) United States
(12) Patent Application Publication
(10) Pub. No.: US 2023/0026189 A1
(43) Pub. Date: Jan. 26, 2023

(54) CELL AGGREGATE INTERNAL PREDICTION METHOD, COMPUTER READABLE MEDIUM, AND IMAGE PROCESSING DEVICE

(71) Applicant: EVIDENT CORPORATION, Nagano (JP)

(72) Inventors: Yoko KAMATO, Tokyo (JP); Elena SHIMOJI, Tokyo (JP); Takuma DEZAWA, Tokyo (JP)

(73) Assignee: EVIDENT CORPORATION, Nagano (JP)

(21) Appl. No.: 17/687,916

(22) Filed: Mar. 7, 2022

(30) Foreign Application Priority Data
Jul. 20, 2021 (JP) 2021-119597

(51) Int. Cl. G01N 33/483; G06T 7/00; G06T 7/60; G01N 33/50

(52) U.S. Cl. CPC G01N 33/4833; G06T 7/0012; G06T 7/60; G01N 33/5008; G06T 2207/10101; G06T 2207/30024; H04N 13/111

(57) ABSTRACT

An internal prediction method includes acquiring an image of a cell aggregate, calculating a feature amount related to a shape of the cell aggregate on the basis of the image, and outputting structure information related to an internal structure of the cell aggregate on the basis of the feature amount.

### CROSS REFERENCE TO RELATED APPLICATIONS

This application is based upon and claims the benefit of priority from prior Japanese Patent Application No. 2021-119597, filed Jul. 20, 2021, the entire contents of which are incorporated herein by this reference.

### TECHNICAL FIELD

The disclosure of the present specification relates to a cell aggregate internal prediction method, a computer readable medium, and an image processing device.

### BACKGROUND

A technique for stable supplying a large number of cells while maintaining the quality at a certain level or higher is essential to promote drug discovery and regenerative medicine using pluripotent stem cells. Thus, in recent years, suspension culture capable of culturing a larger number of cells at a time than monolayer culture is gaining attention.

Unlike the monolayer culture in which cells are planarly cultured, the suspension culture produces a cell aggregate by three-dimensionally culturing cells. The cells in the cell aggregate act by interacting with the surrounding cells or the like in the same manner as in vivo. Thus, for example, for evaluating drug efficacy, using the cell aggregate cultured in the suspension culture makes it possible to perform accurate evaluation under conditions closer to in vivo than using the cells cultured in the monolayer culture. Such a technique related to the drug efficacy evaluation is described in, for example, JP 2015-181348 A.

### SUMMARY

An internal prediction method according to an aspect of the present invention includes acquiring an image of a cell aggregate, calculating a feature amount related to a shape of the cell aggregate on the basis of the image, and outputting structure information related to an internal structure of the cell aggregate on the basis of the feature amount.

A non-transitory computer readable medium according to an aspect of the present invention stores an internal prediction program of a cell aggregate, in which the program causes a computer to execute processes of acquiring an image of the cell aggregate, calculating a feature amount related to a shape of the cell aggregate on the basis of the image, and outputting structure information related to an internal structure of the cell aggregate on the basis of the feature amount.

An image processing device according to an aspect of the present invention includes an acquisition portion that acquires an image of a cell aggregate, a calculation portion that calculates a feature amount related to a shape of the cell aggregate on the basis of the image, and an output portion that outputs structure information related to an internal structure of the cell aggregate on the basis of the feature amount.

### BRIEF DESCRIPTION OF DRAWINGS

The present invention will be more apparent from the following detailed description when the accompanying drawings are referenced.

FIG. 1 is a diagram illustrating an example of a configuration of a system;

FIG. 2 is a diagram illustrating an example of a configuration of a microscope;

FIG. 3 is a diagram illustrating an example of a functional configuration of a server device;

FIG. 4 is a diagram illustrating an example of a flowchart of internal prediction processing according to a first embodiment;

FIG. 5 is a diagram illustrating an example of a flowchart of feature amount calculation processing;

FIG. 6 is diagram describing image selection processing;

FIG. 7 is diagram describing contour extraction processing and the feature amount calculation processing;

FIG. 8 is diagram illustrating an example of detecting abnormality of a cell aggregate on the basis of a second feature amount;

FIG. 9 is diagram illustrating an example of detecting abnormality of a cell aggregate on the basis of a first feature amount;

FIG. 10 is diagram illustrating an example of a database of an internal structure of the cell aggregate;

FIG. 11 is diagram illustrating an example of an output of a prediction result of the internal structure of the cell aggregate;

FIG. 12 is diagram illustrating another example of a database of an internal structure of the cell aggregate;

FIG. 13 is diagram illustrating another example of an output of a prediction result of the internal structure of the cell aggregate;

FIG. 14 is a diagram illustrating an example of a flowchart of internal prediction processing according to a second embodiment;

FIG. 15 is diagram illustrating a display example of a contour of the cell aggregate;

FIG. 16 is diagram illustrating a correction example of the contour of the cell aggregate;

FIG. 17 is diagram illustrating still another example of an output of a prediction result of the internal structure of the cell aggregate; and

FIG. 18 is diagram illustrating an example of a hardware configuration of a computer to achieve the server device.

### DESCRIPTION OF EMBODIMENTS

It is difficult to observe an internal structure of a cell aggregate during cell culture from the outside, and observable parts are limited to a part of the cell aggregate. Thus, it is difficult to determine whether the cell culture is proceeded properly in suspension culture as compared with monolayer culture in which the whole cells can be observed.

Considering such circumstances, an embodiment of the present invention will be described hereinafter.

### First Embodiment

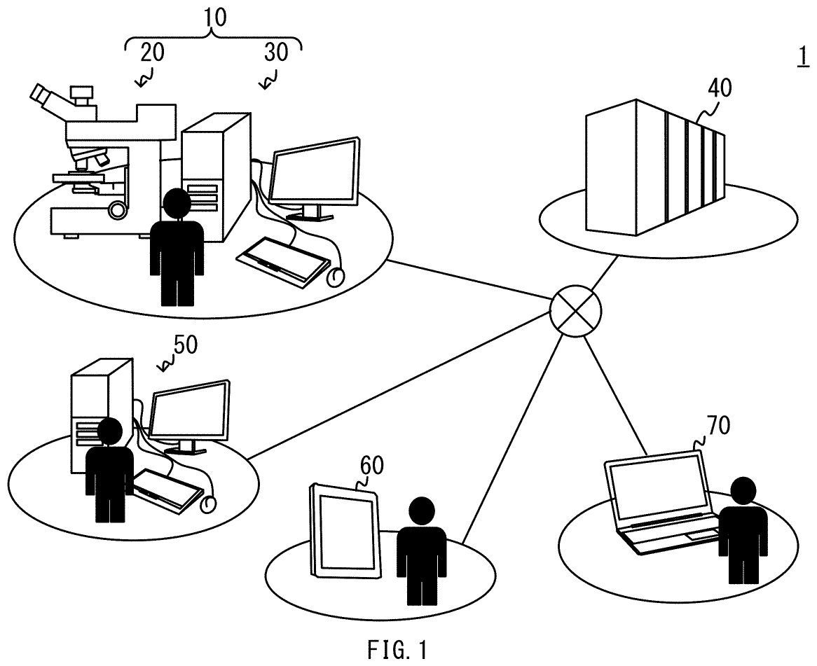

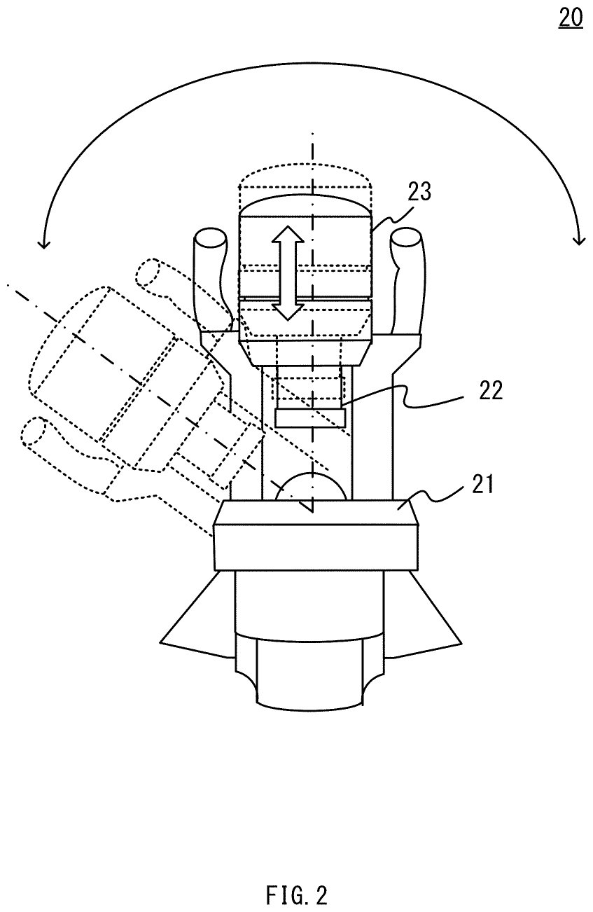

FIG. 1 is a diagram illustrating an example of a configuration of a system 1. FIG. 2 is a diagram illustrating an example of a configuration of a microscope 20. The system 1 is a system for observing a cell aggregate such as a spheroid produced by culturing cells by suspension culture and predicting an internal structure of the cell aggregate. Hereinafter, a configuration of the system 1 will be described with reference to FIG. 1 and FIG. 2.

The system 1 includes a microscope system 10, a server device 40, and a plurality of client devices (a client device 50, a client device 60, and a client device 70), which are communicatively connected to one another through a network.

Note that a type of the network connecting between the devices is not particularly limited. For example, the network may be a public network such as an internet, a dedicated network, or a LAN (local area network). The connection between the devices may be wired connection or wireless connection.

The microscope system 10 includes a microscope 20 which captures an image of the cell aggregate and a control device 30 which controls the microscope 20. The control device 30 controls the microscope 20, so that the microscope 20 captures an image of the cell aggregate taken out from a culture environment, and, further, the control device 30 sends the image of the cell aggregate thus generated to the server device 40.

The microscope 20 is only required to include an imaging function for capturing an image of the cell aggregate. FIG. 1 shows an example in which the microscope system 10 includes the microscope 20 with an eyepiece. However, the microscope 20 may be a digital microscope without an eyepiece. The microscope 20 desirably has a structure capable of freely changing the direction of an objective lens 22 and a digital camera 23 with respect to a stage 21 as shown in FIG. 2. Further, it desirably has a function of repeating the movement of the focal plane of the objective lens 22 in the optical axis direction and the image capturing. That is, the microscope 20 desirably has a configuration capable of capturing an image of the cell aggregate from a variety of directions at a variety of depths.

Examples of an observation method in which the microscope 20 is used include a bright field observation method and a phase difference observation method. However, as described below, the microscope 20 is only required to acquire an image in which at least a contour of the cell aggregate can be recognized, and thus it may be used in any observation method other than the above.

The server device 40 is an image processing device which executes internal prediction processing described below on the basis of the image of the cell aggregate. The server device 40 acquires the image of the cell aggregate generated by the microscope system 10, predicts the internal structure of the cell aggregate on the basis of the image, and outputs a prediction result. More specifically, the server device 40 predicts the internal structure on the basis of a shape feature of the cell aggregate appeared in the image of the cell aggregate.

The client devices (the client device 50, the client device 60, and the client device 70) acquire the prediction result outputted by the control device 30 by responding to a request from a user and display it on a display device. Thus, the client device is only required to include at least an input device which receives the request from the user, the display device which displays the prediction result, and a communication device which communicates with the server device 40. Note that the control device 30 may be operated as the client device and output the prediction result on the display device (a display portion) included in the control device 30. That is, the control device 30 may output the prediction result by displaying the prediction result by itself.

Note that, the client device may be, for example, a desktop computer such as the client device 50, a tablet computer such as the client device 60, or a laptop computer such as the client device 70. Further, it may be a smartphone, a cellular phone, or the like. Further, each client device may be a dedicated terminal for a specific user or a shared terminal shared by multiple users.

According to the system 1 configured as described above, the user can easily recognize the internal structure of the cell aggregate by confirming the prediction result displayed on the client device. Thus, when the cell aggregates are periodically sampled to acquire the images of the cell aggregates during the cell culture, it becomes possible to detect abnormality of the cell culture at an early stage and efficiently culture the cells without performing useless culture.

Further, the system 1 predicts the internal structure of the cell aggregate from the shape feature of the cell aggregate. Thus, a high-performance device for visualizing in detail the inside of the cell aggregate which is three-dimensionally grown is not necessarily required, and many existing microscope systems can be used as the imaging device. Further, since it is only required to achieve an image quality enough to extract the contour, an imaging time can be shortened. This makes it possible to obtain the prediction result in a short time and allows the user to recognize the culture state without delay.

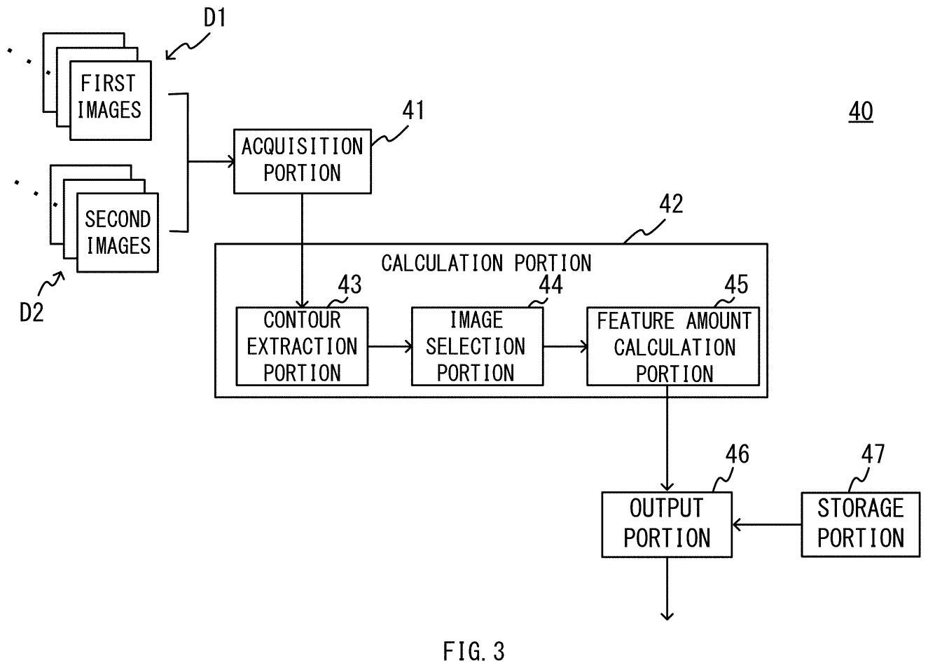

FIG. 3 is a diagram illustrating an example of a functional configuration of a server device 40. As shown in FIG. 3, the server device 40 includes at least an acquisition portion 41 which acquires an image of the cell aggregate, a calculation portion 42 which calculates a feature amount related to a shape of the cell aggregate, and an output portion 46 which outputs structure information related to the internal structure of the cell aggregate. The server device 40 may further include a storage portion 47 in which a database described below is constructed. Hereinafter, the functional configuration of the server device 40 related to a prediction processing method which predicts the internal structure of the cell aggregate will be described with reference to FIG. 3.

The acquisition portion 41 acquires, for example, the image of the cell aggregate generated by the microscope system 10. The acquisition portion 41 desirably acquires two or more images of the cell aggregate captured from mutually different directions. Using the images captured from the different directions facilitates recognition of the whole shape of the cell aggregate in the calculation portion 42 described below as compared with the case of using only the image captured from one direction. Further, the acquisition portion 41 further desirably acquires a plurality of the images obtained by imaging mutually different surfaces of the cell aggregate in each imaging direction. Acquiring the plurality of the images captured in the same direction makes it possible to select the image suitable for recognizing the shape of the cell aggregate in each imaging direction. This further facilitates the recognition of the whole shape of the cell aggregate in the calculation portion 42. Further, the different directions are desirably directions that intersect with each other. Using the intersecting directions makes it possible to obtain the images of the cell aggregate captured at different angles with respect to the gravity direction. For example, the acquisition portion 41 may acquire a plurality of first images D1 obtained by imaging the mutually different surfaces of the cell aggregate from a vertical direction (a first direction) and a plurality of second images D2 obtained by imaging the mutually different surfaces of the cell aggregate from a horizontal direction (a second direction). Note that, for example, the images of the cell aggregate may be previously stored in the storage portion 47 of the server device 40, and the acquisition portion 41 may read the images from the storage portion 47.

The calculation portion 42 calculates the feature amount related to the shape of the cell aggregate on the basis of the images acquired by the acquisition portion 41. The calculation portion 42 may include, for example, a contour extraction portion 43, an image selection portion 44, and a feature amount calculation portion 45.

The contour extraction portion 43 specifies a contour of the cell aggregate on the basis of the images acquired by the acquisition portion 41. A method for extracting and specifying the contour is not particularly limited. Any existing extraction method can be adopted. In a case where the plurality of the images are acquired by the acquisition portion 41, the contour extraction portion 43 desirably specifies the contour of the cell aggregate in each of the images thus acquired. For example, in a case where the plurality of the first images and the plurality of the second images are acquired by the acquisition portion 41, the contour extraction portion 43 desirably specifies the contour of the cell aggregate in each of the plurality of the first images and specifies the contour of the cell aggregate in each of the plurality of the second images.

The image selection portion 44 selects the image to be used for the feature amount calculation on the basis of the contour specified by the contour extraction portion 43. The image selection portion 44 desirably selects the image in each imaging direction and, further, desirably selects the image having the maximum contour among the images in the same imaging direction. That is, it is desirable to select the image having the maximum contour in each imaging direction. For example, in a case where the plurality of the first images and the plurality of the second images are acquired by the acquisition portion 41, the image selection portion 44 selects a third image on the basis of a plurality of the contours corresponding to the plurality of the first images and selects a fourth image on the basis of a plurality of the contours corresponding to the plurality of the second images. The image selection portion 44 desirably selects the image having the maximum contour as the third image among the plurality of the first images and selects the image having the maximum contour as the fourth image among the plurality of the second images. Note that the image selection portion 44 may select the image, for example, by defining the contour in which a partitioned region has the maximum area as the maximum contour.

The feature amount calculation portion 45 calculates the feature amount related to the shape of the cell aggregate on the basis of the image selected by the image selection portion 44. The feature amount calculation portion 45 desirably calculates the feature amount in each imaging direction and thus desirably calculates the feature amount in each image selected by the image selection portion 44. For example, in a case where the image selection portion 44 selects the third image from the plurality of the first images and the fourth image from the plurality of the second images, the feature amount calculation portion 45 desirably calculates the feature amount on the basis of each of the third image and the fourth image.

The feature amount calculated by the feature amount calculation portion 45 is a feature amount related to the shape of the cell aggregate recognizable from the contour of the cell aggregate. The feature amount calculated by the feature amount calculation portion 45 desirably includes at least one of the feature amount (hereinafter, referred to as a first feature amount) related to unevenness on the surface of the cell aggregate and the feature amount (hereinafter, referred to as a second feature amount) related to deviation from the ideal shape of the cell aggregate. Note that the ideal shape of the cell aggregate is, for example, a spherical shape, and the ideal shape appeared in the image is, for example, a circular shape.

The output portion 46 outputs the structure information related to the internal structure of the cell aggregate on the basis of the feature amount calculated by the feature amount calculation portion 45. The output portion 46 desirably refers to a database in which the structure information related to the internal structure of the cell aggregate is associated with the feature amount. For example, the output portion 46 desirably acquires the structure information related to the internal structure of the cell aggregate form the database constructed in the storage portion 47 using the feature amount calculated by the feature amount calculation portion 45 and outputs the structure information thus acquired. That is, the storage portion 47 stores the feature amount and the structure information in association with each other. Note that the database may be constructed in a device different from the server device 40.

Information related to the internal structure of the cell aggregates is collected by observing a number of the cell aggregates in detail in advance and recorded in the database as the structure information. The structure information recorded in the database may be, for example, information generated on the basis of a tomographic image of the cell aggregate acquired by optical coherence tomography (OCT), information generated on the basis of a tomographic image of the cell aggregate acquired by a fluorescence observation method, or information generated on the basis of an image obtained by actually cutting the cell aggregate and imaging a resulting cross section. The information described above may be an image itself obtained by imaging the cell aggregate or a model image showing a distribution of cells in the cell aggregate generated from the image, as long as the information is associated with the feature amount.

The server device 40 configured as described above executes the internal prediction processing described below. When the quality of the cell aggregate is deteriorated due to weakening of the cells or the like, the bond between the cells is also weakened, and the whole shape of the cell aggregate starts to collapse. Thus, when the cell aggregate is not normal, the shape of the cell aggregate is deviated from the ideal shape, and, further, the unevenness on the surface becomes evident. The server device 40 can detect a slight difference in the shape of the cell aggregate hardly recognizable by human eyes by quantifying the shape of the cell aggregate as the feature amount. Then, by referring to the database on the basis of the shape of the cell aggregate thus detected, the server device 40 can predict the internal structure of the cell aggregate with high accuracy. Thus, according to the server device 40 and the internal prediction method performed by the server device 40 described above, it becomes possible to easily recognize the internal structure of the cell aggregate from the image of the cell aggregate and detect abnormality of the cell culture at an early stage.

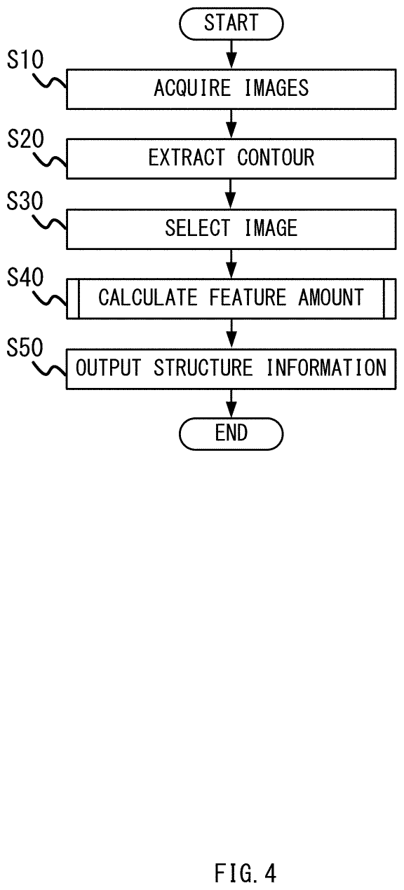

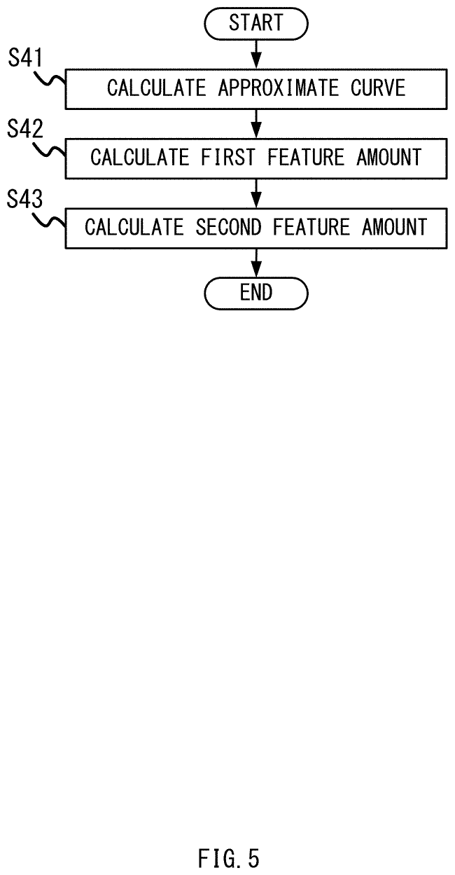

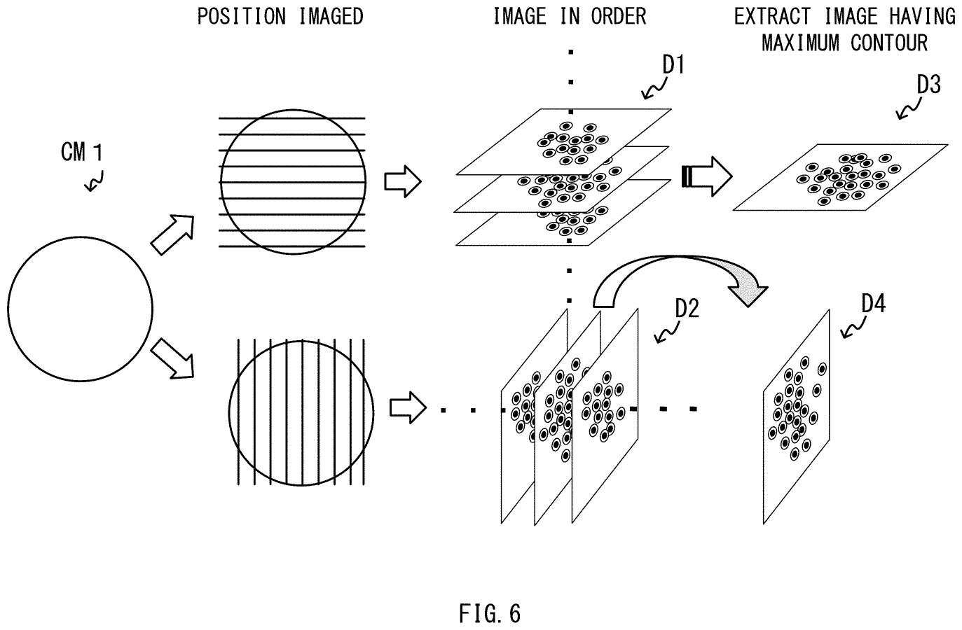

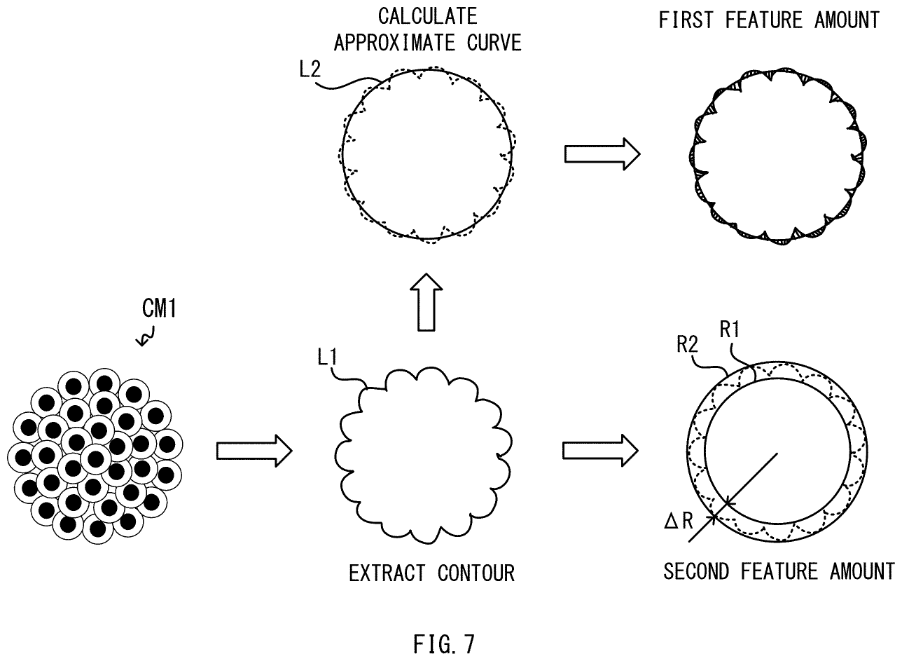

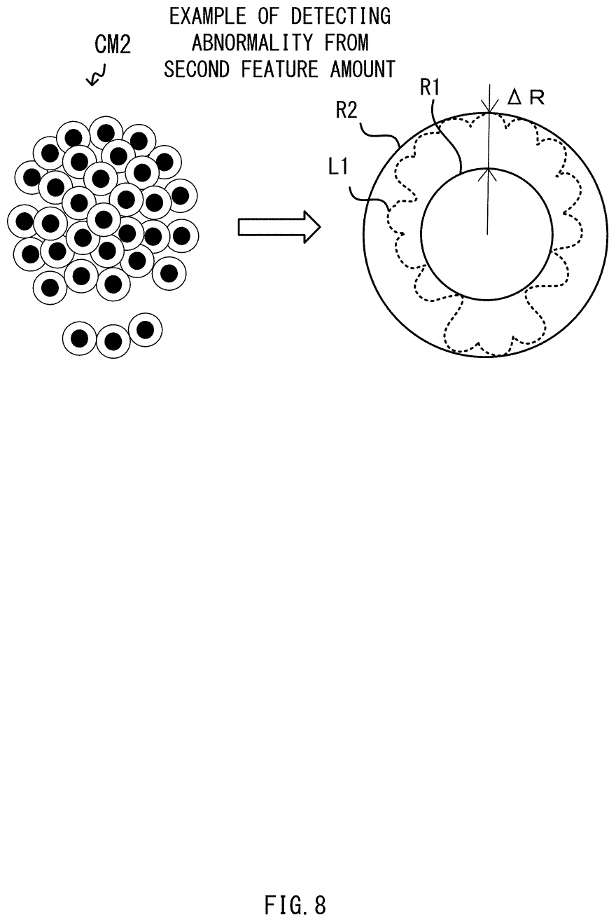

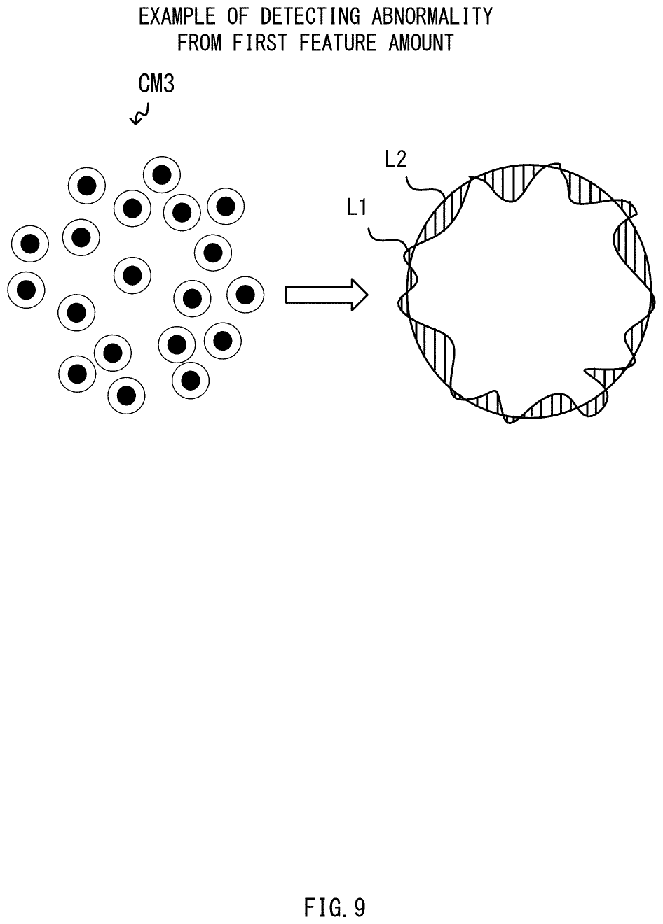

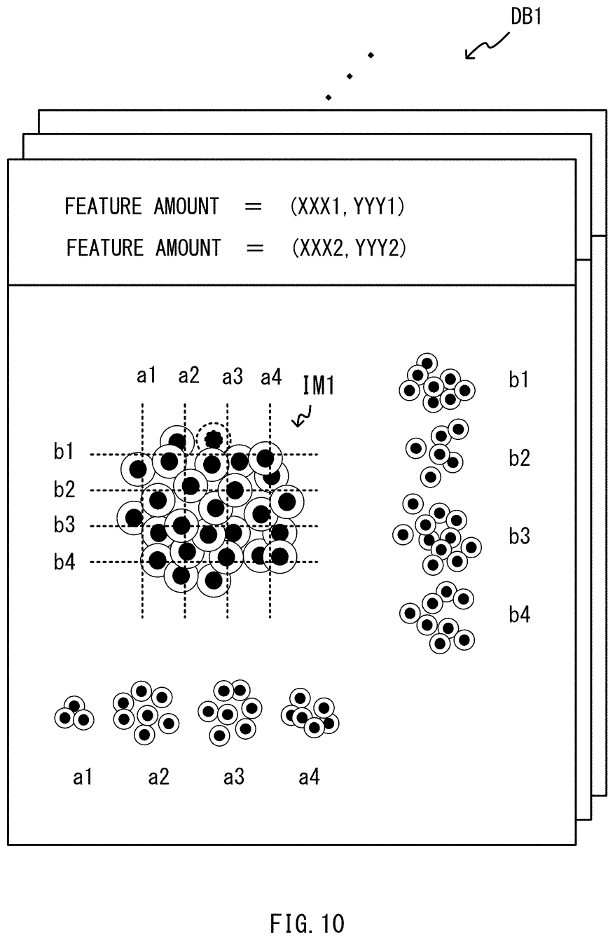

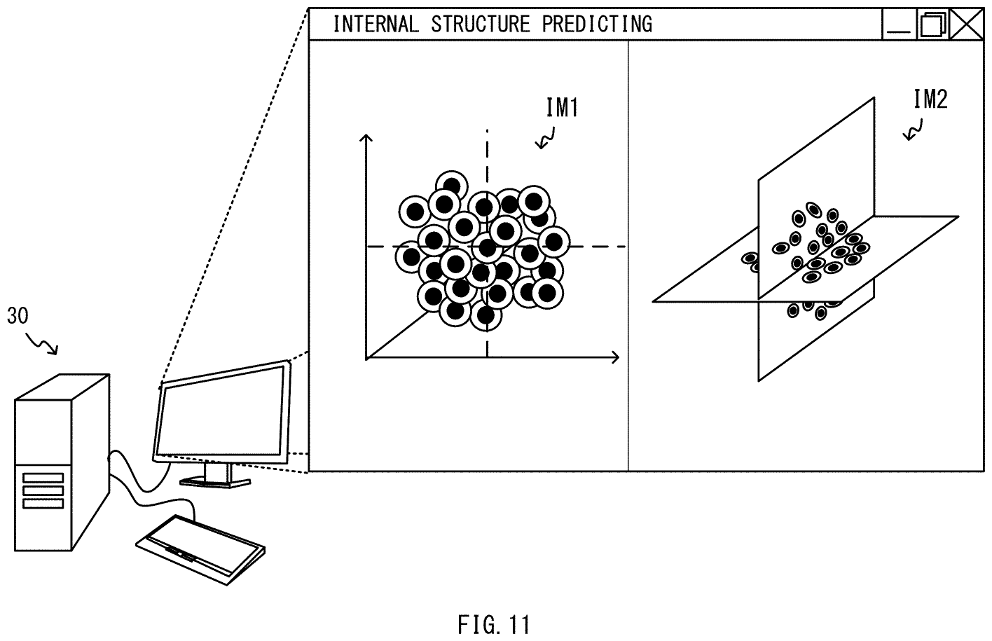

FIG. 4 is a diagram illustrating an example of a flowchart of internal prediction processing according to the present embodiment. FIG. 5 is a diagram illustrating an example of a flowchart of feature amount calculation processing. FIG. 6 is diagram describing image selection processing. FIG. 7 is diagram describing contour extraction processing and the feature amount calculation processing. FIG. 8 is diagram illustrating an example of detecting abnormality of a cell aggregate on the basis of a second feature amount. FIG. 9 is diagram illustrating an example of detecting abnormality of a cell aggregate on the basis of a first feature amount. FIG. 10 is diagram illustrating an example of a database of an internal structure of the cell aggregate. FIG. 11 is diagram illustrating an example of an output of a prediction result of the internal structure of the cell aggregate. Hereinafter, the internal prediction processing which predicts the internal structure of the cell aggregate performed by the server device 40 will be described in detail with reference to FIG. 4 to FIG. 11.

Below, a case where the images of the cell aggregate as a prediction object captured by the microscope system 10 are stored in advance in the server device 40 will be described as an example. In this example, the images stored in the server device 40 include the plurality of the first images D1 obtained by imaging the cell aggregate from the vertical direction and the plurality of the second images D2 obtained by imaging the cell aggregate from, for example, the horizontal direction. Further, the plurality of the first images D1 are the images of the cell aggregate corresponding to mutually different focal planes and the plurality of the second images D2 are also the images of the cell aggregate corresponding to mutually different focal planes.

The server device 40 executes a predetermined program and starts the internal prediction processing shown in FIG. 4, for example, by responding to a request from the client device. Here, a case where the control device 30 requests the internal prediction of the cell aggregate to the server device 40 as the client device will be described as an example.

Upon receiving the request from the control device 30, the server device 40 first acquires images of a cell aggregate CM1 as a prediction object (Step S10). In this step, the acquisition portion 41 acquires the plurality of the first images D1 and the plurality of the second images D2 from the storage portion 47. The plurality of the first images D1 are, for example, as shown in FIG. 6, images obtained by imaging the cell aggregate CM1 at different positions (surfaces) from the vertical direction, while the plurality of the second images D2 are, for example, as shown in FIG. 6, images obtained by imaging the cell aggregate CM1 at different positions (surfaces) from the horizontal direction. Note that, in FIG. 6, the cells constituting the cell aggregate are clearly appeared in the first images D1 and the second images D2. However, the first images D1 and the second images D2 are only required to include information necessary for specifying the contour of the cell aggregate.

The images acquired in the Step S10 are not limited to the images acquired from the vertical direction and the horizontal direction. However, including the images captured from the vertical direction and the horizontal direction means including the images captured in a direction (the horizontal direction) largely affected by the gravity and the images captured in a direction (the vertical direction) less affected by the gravity, thereby providing an advantage that a degree of deterioration of the cell aggregate can be easily recognized. Note that the images acquired in the Step S10 may include images captured from three or more directions or images captured from two reversely directed directions.

When the images are acquired, the server device 40 extracts the contour of the cell aggregate on the basis of the images thus acquired (Step S20). In this step, the contour extraction portion 43 extracts the contour of the cell aggregate from each image acquired in the Step S10.

Further, the server device 40 selects the image used for the feature amount calculation on the basis of the contour extracted in the Step S20 (Step S30). In this step, the image selection portion 44 specifies the maximum contour in each imaging direction and selects the image having the maximum contour. That is, as shown in FIG. 6, the image selection portion 44 selects the third image D3 having the maximum contour from the plurality of the first images D1 and the fourth image D4 having the maximum contour from the plurality of the second images D2.

Subsequently, the server device 40 executes the feature amount calculation processing shown in FIG. 5 (Step S40). In the feature amount calculation processing, the feature amount calculation portion 45 first calculates an approximate curve (Step S41). In the Step S41, for example, as shown in FIG. 7, the feature amount calculation portion 45 calculates, on the basis of a contour L1 of the cell aggregate CM1 extracted from the image selected in the Step S30, an approximate curve L2 approximating the contour L1. The approximate curve L2 is calculated in order to express the whole shape of the cell aggregate CM1, and, for calculating the first feature amount related to the unevenness on the surface of the cell aggregate, the approximate curve L2 is used as a reference surface with respect to the unevenness on the surface. Thus, it is not necessary to perform the approximation with an excessively high-order function, and, for example, the approximation may be performed with an equation of circle or ellipse.

After calculating the approximate curve, the feature amount calculation portion 45 calculates the first feature amount on the basis of the contour L1 and the approximate curve L2 calculated in the Step S41 (Step S42). In this step, for example, as shown in FIG. 7, the feature amount calculation portion 45 calculates an area of a region surrounded by the contour L1 and the approximate curve L2 as the first feature amount, thereby quantifying an amount of the unevenness caused on the surface of the cell aggregate.

Further, the feature amount calculation portion 45 calculates the second feature amount related to deviation from the ideal shape of the cell aggregate on the basis of the contour L1 (Step S43). In this step, for example, as shown in FIG. 7, the feature amount calculation portion 45 calculates the second feature amount using a difference ΔR in the radius of a circle R1 inscribed in the contour L1 and a circle R2 circumscribing the contour L1. Note that the second feature amount may be, for example, roundness representing a degree of deviation from a circle shape.

The calculation methods of the first feature amount and the second feature amount are not limited to the above examples. For example, the second feature amount is only required to indicate a deviation degree from the ideal shape and thus may be calculated on the basis of the approximate curve instead of the roundness. For example, in a case where the approximate curve is expressed using an ellipse equation, an ellipticity may be calculated as the second feature amount instead of the roundness.

Both the first feature amount and the second feature amount are suitable parameters for detecting abnormality of the cell aggregate. Predicting the internal structure from these parameters makes it possible to find abnormality of the cell aggregate at an early stage. Specifically, when the second feature amount indicating the deviation from the ideal shape such as the roundness is used, it becomes possible to quantitatively recognize a state where the shape of the cell aggregate collapses by the influence of the gravity or the like, for example, as seen in a cell aggregate CM2 shown in FIG. 8, resulting from weakening of the bonding force caused by deterioration of the cell aggregate CM2. Further, when the first feature amount indicating the unevenness on the surface is used, it becomes possible to quantitatively recognize a state where the rough unevenness is generated on the surface, for example, due to dispersing of the cells caused by weakening of the bond between the cells, as seen in a cell aggregate CM3 shown in FIG. 9. Thus, it becomes possible to detect abnormality of the cell aggregate CM3 which appears to maintain the ideal shape when judged only by the second feature amount.

After ending the feature amount calculation processing, the server device 40 outputs the structure information related to the internal structure of the cell aggregate on the basis of the feature amount thus calculated (Step S50). In this step, the output portion 46 acquires the structure information associated with the first feature amount calculated in the Step S42 and the second feature amount calculated in the Step S43 by referring to a database DB1 constructed in the storage portion 47. As shown in FIG. 10, the database DB1 stores, for example, a model image IM1 showing a distribution of the cells in the cell aggregate in association with the feature amount (two combinations of the first feature amount and the second feature amount). Further, the database DB1 stores the tomographic image of each of the cross sections (cross sections a1 to a4 and cross sections b1 to b4) of the model image IM1 which is a 3D image.

After the output portion 46 outputs the structure information acquired from the storage portion 47 to the control device 30, the server device 40 ends the internal prediction processing shown in FIG. 4. Note that, after receiving the structure information from the server device 40, the control device 30 displays the structure information as a prediction result of the internal structure of the cell aggregate as shown in FIG. 11. FIG. 11 shows a state where the model image IM1 and a tomographic image IM2 acquired from the database DB1 are displayed as the prediction result of the internal structure. Note that the tomographic image IM2 is, for example, an image obtained by combining the tomographic images at the positions corresponding to the third image and the fourth image used for calculating the feature amount.

As described above, the server device 40 outputs the prediction result of the internal structure of the cell aggregate by executing the internal prediction processing shown in FIG. 4. In this manner, the user can easily recognize the internal structure of the cell aggregate on the basis of the prediction result displayed on the client device, making it possible to detect abnormality of the cell aggregate at an early stage. In particular, even when the internal structure of the cell aggregate is not appeared in the image, the above internal prediction processing can predict the internal structure as long as the contour can be recognized. Thus, the user can recognize the internal structure of the cell aggregate without using a special device.

Note that a display method of the prediction result is not particularly limited. FIG. 10 shows an example of displaying the tomographic image which predicts the cell distribution on the cross sections used for the feature amount calculation, as shown in the tomographic image IM2. However, the server device 40 may output the tomographic image on any cross section specified by the user to the client device. Further, the tomographic images on two or more cross sections may be simultaneously displayed.

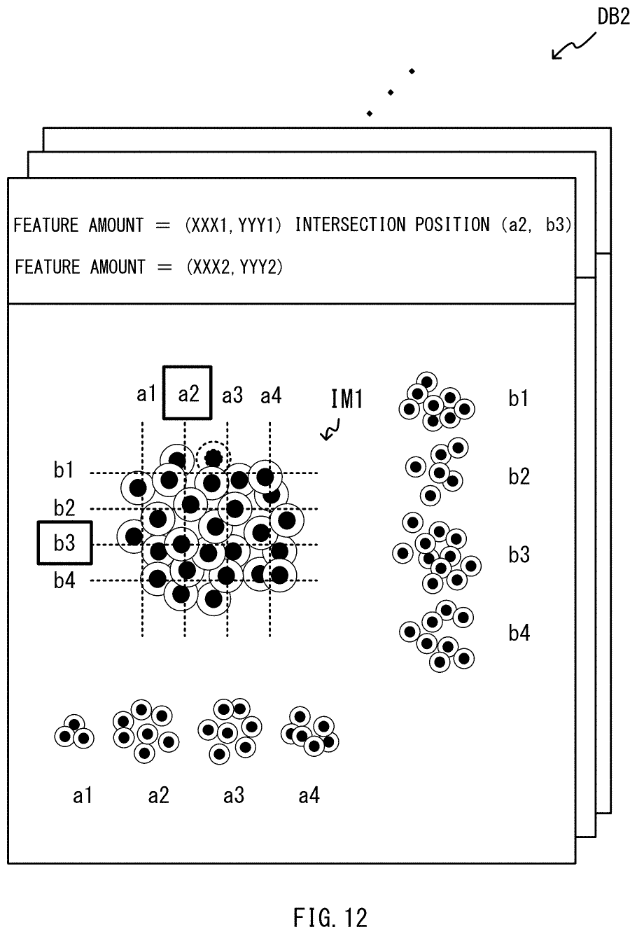

FIG. 12 is diagram illustrating another example of a database of an internal structure of the cell aggregate. FIG. 10 shows an example of storing the structure information in association with the feature amount in the database DB1. However, as shown in a database DB2 in FIG. 12, the structure information may be associated with a combination of the feature amount and an intersection position. Note that the intersection position indicates a positional relationship between the images (the third image and the fourth image) corresponding to the two sets of the feature amount.

Even if the combination of the feature amount at the cross sections having the maximum contour is the same, the whole shape of the cell aggregate may greatly vary depending on the positional relationship between the cross sections. Thus, constructing a database by collecting the structure information in each combination of the feature amount and the intersection position of the cross sections makes it possible to predict the internal structure of the cell aggregate with higher accuracy. Thus, in the Step S50 in FIG. 4, the output portion 46 may acquire the structure information on the basis of the positional relationship of the third image and the fourth image, the feature amount corresponding to the third image, and the feature amount corresponding to the fourth image.

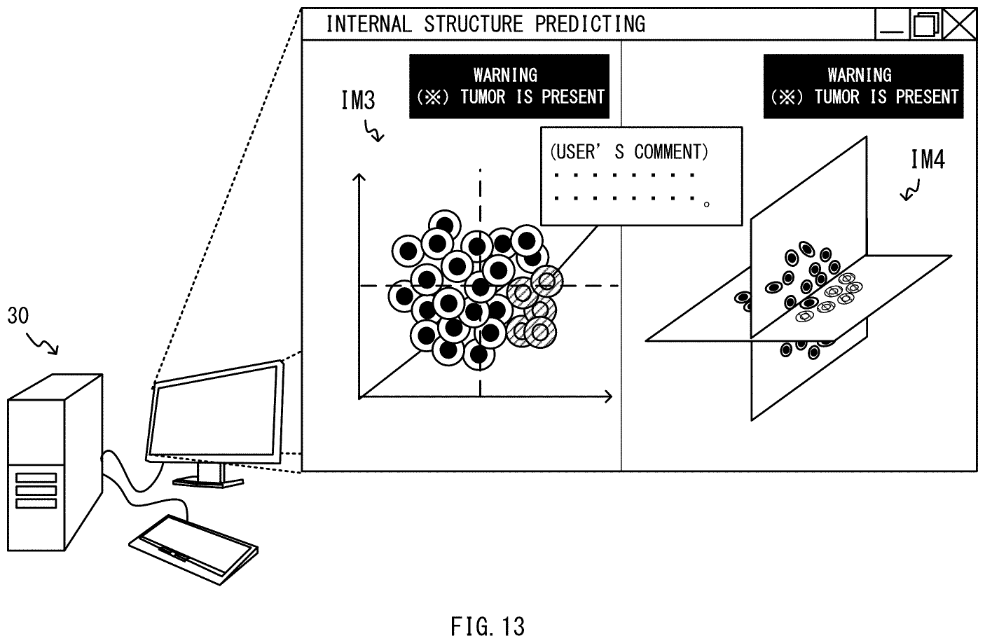

FIG. 13 is diagram illustrating another example of an output of a prediction result of the internal structure of the cell aggregate. FIG. 11 shows an example of displaying the result without classifying the cells distributed in the cell aggregate. However, the cells distributed in the cell aggregate may be classified and displayed. In this case, a classification result in which the cells constituting the cell aggregate are classified is included in the model image or the tomographic image previously stored in the database. In this manner, as shown in FIG. 13, the cells can be displayed while having been classified in a model image IM3 and a tomographic image IM4. Note that FIG. 13 shows an example where tumor cells and normal cells are distinguished from each other and displayed. Further, as shown in FIG. 13, the presence of the tumor cells may be emphasized to give the user a warning. Further, information such as opinions of other users on the cell aggregate may be shared by simultaneously displaying the model image IM3 and comments on the model image IM3 attached by other users.

### Second Embodiment

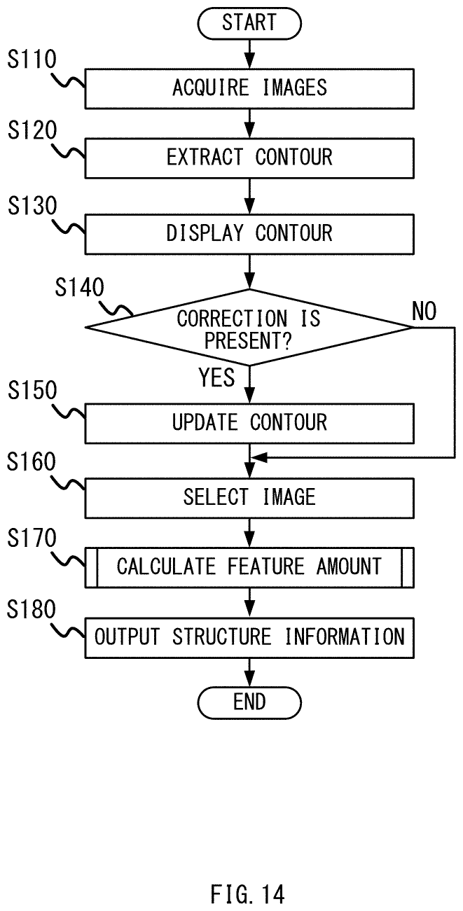

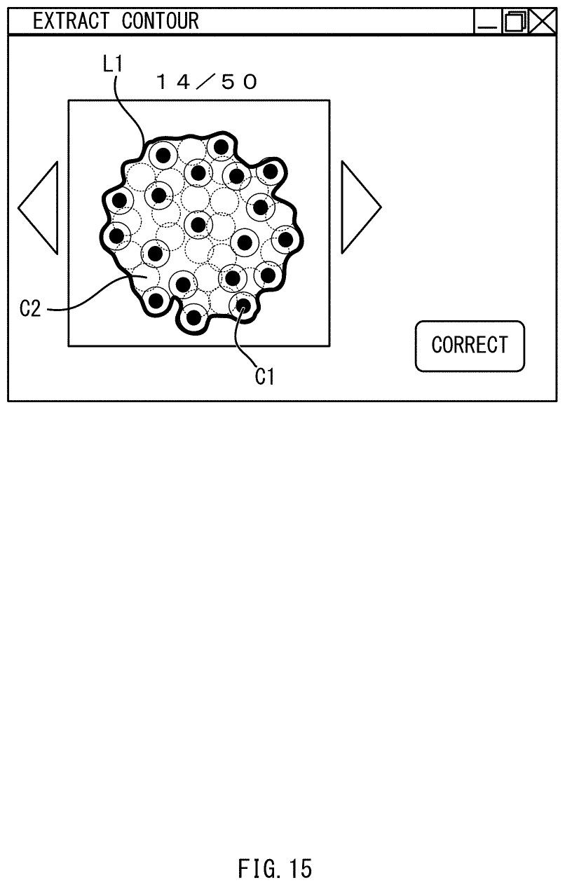

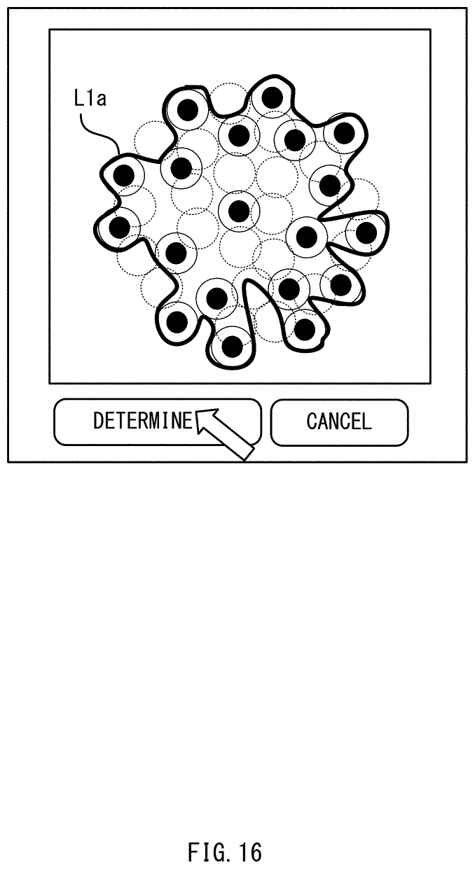

FIG. 14 is a diagram illustrating an example of a flowchart of internal prediction processing according to the present embodiment. FIG. 15 is diagram illustrating a display example of a contour of the cell aggregate. FIG. 16 is diagram illustrating a correction example of the contour of the cell aggregate. Hereinafter, the internal prediction processing according to the present embodiment will be described in detail with reference to FIG. 14 to FIG. 16. Note that the system according to the present embodiment has the same configuration as that of the system 1 according to the first embodiment. Thus, each constituent element is referred to with the same reference sign as in the first embodiment. Further, like the internal prediction processing according to the first embodiment, the internal prediction processing according to the present embodiment is performed by the server device 40.

The server device 40 executes a predetermined program and starts the internal prediction processing shown in FIG. 14, for example, by responding to a request from the client device. Here, like the first embodiment, a case where the control device 30 requests the internal prediction of the cell aggregate to the server device 40 as the client device will be described as an example.

After receiving the request from the control device 30, the server device 40 first acquires the image of the cell aggregate as a prediction object (Step S110) and extracts the contour of the cell aggregate on the basis of the image thus acquired (Step S120). The processing in the Step S110 and the Step 120 is the same as that in the Step S10 and the Step 20 shown in FIG. 4.

After extracting the contour, the server device 40 displays the contour on the display device. In this step, the server device 40 may display an image obtained by, for example, as shown in FIG. 15, superimposing the contour L1 calculated in the Step S120 on the image acquired in the Step S110 on the control device 30, so that the user can recognize the contour L1 recognized by the server device 40.

Further, the server device 40 determines the presence or absence of a correction instruction (Step S140), and, if the correction instruction is inputted (Step S140: YES), the contour of the cell aggregate is updated in accordance with the correction instruction (Step S150). For example, the server device 40 may receive the correction of the contour L1 extracted in the Step S120 when a correction button shown in FIG. 15 is pressed down by the user, and the contour of the cell aggregate may be updated from the contour L1 to a contour L1a corrected by the user using GUI when a determination button shown in FIG. 16 is pressed down by the user.

Note that a cell C1 and a cell C2 shown in FIG. 15 and FIG. 16 are a cell present on the focal plane and a cell present in front of or behind the focal plane, respectively. FIG. 15 and FIG. 16 show an example where the contour of the cell aggregate on the focal plane is more correctly recognized by the server device 40 when the user defines the contour by avoiding the cell C2 present in front of or behind the focal plane.

Subsequently, the server device 40 selects the image to be used for the feature amount calculation (Step S160), executes the feature amount calculation processing on the basis of the image thus selected (Step S170), and outputs the structure information related to the internal structure of the cell aggregate on the basis of the feature amount thus calculated (Step S180). The processing from the Step S160 to the Step S180 is the same as that from the Step S30 to the Step S50 shown in FIG. 4. However, if the contour is updated in the Step S150, the image is selected on the basis of the updated contour instead of the contour before the update in the Step 160, and the feature amount is calculated on the basis of the selected image. That is, the feature amount is calculated on the basis of the updated contour.

As described above, even in the case where the server device 40 executes the internal prediction processing shown in FIG. 14, the prediction result of the internal structure of the cell aggregate is outputted. Thus, like the first embodiment, also in the present embodiment, the user can easily recognize the internal structure of the cell aggregate on the basis of the prediction result displayed on the client device, making it possible to detect abnormality of the cell aggregate at an early stage.

Further, in the present embodiment, the contour of the cell aggregate recognized by the server device 40 can be manually corrected by the user. Adding the judgement of the user in the contour extraction makes it possible to perform the contour extraction with higher accuracy. This makes it possible to calculate the feature amount of the cell aggregate calculated on the basis of the contour with higher accuracy, leading to expectations of an improvement in prediction accuracy of the internal structure.

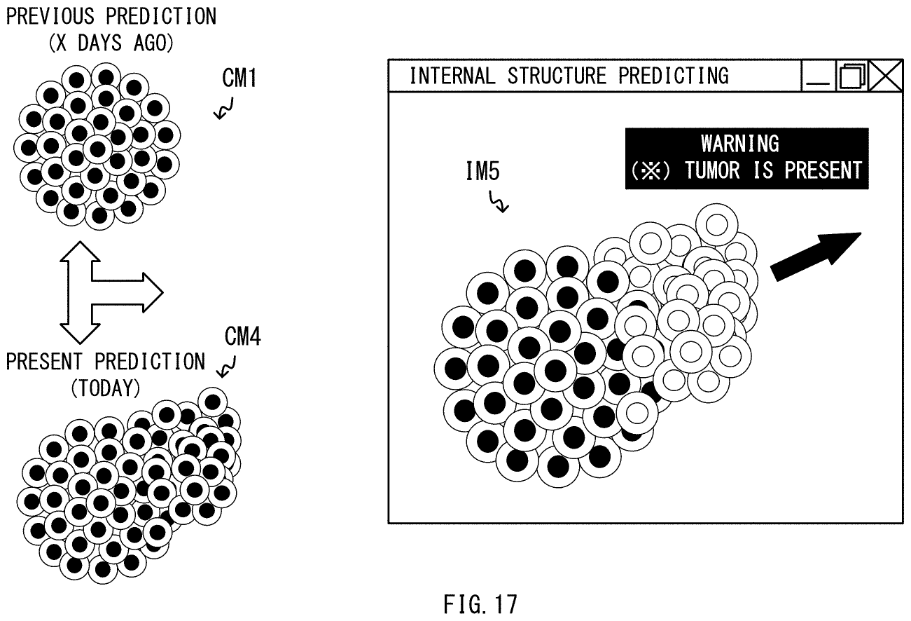

The above description shows an example of predicting the internal structure of the cell aggregate on the basis of the image of the cell aggregate at a certain time point. However, for example, in a case where the internal structure is repeatedly predicted by continuously observing the same cell aggregate, as shown in FIG. 17, the past prediction may be used in the latest prediction.

For example, in a case where the cell aggregate CM1 at the previous prediction time point is grown to a cell aggregate CM4 at the present prediction time point, when a model image IM5 is displayed as the present prediction result as shown in FIG. 17, a part where the growth is abnormally progressed as compared with the previous prediction result is specified and such a part may be classified as tumor cells. Using the past prediction result in this manner makes it possible to display the cells while having been classified even in a case where the cells distributed in the cell aggregate are previously recorded in the database without being classified.

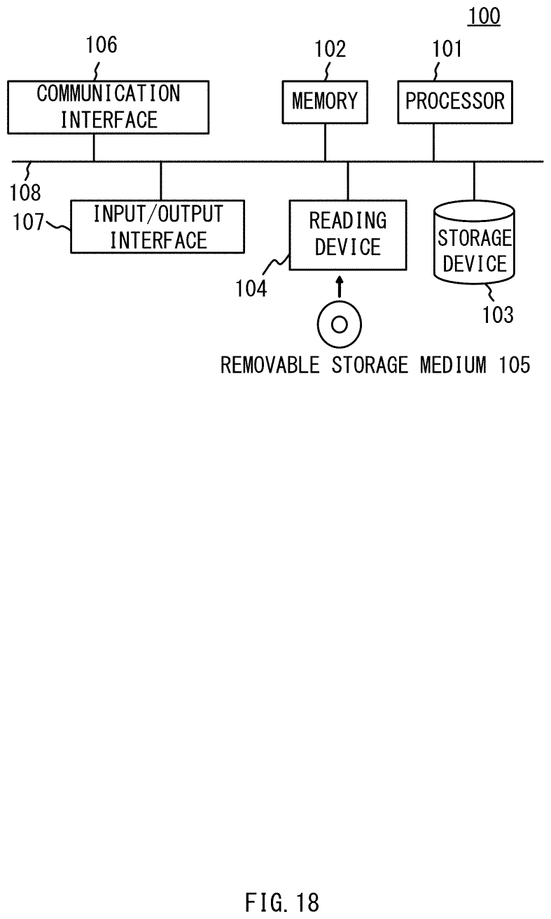

FIG. 18 is diagram illustrating an example of a hardware configuration of a computer 100 to achieve the server device 40 according to the above-described embodiment. As shown in FIG. 18, a computer 100 includes, as a hardware configuration, a processor 101, a memory 102, a storage device 103, a reading device 104, a communication interface 106, and an input/output interface 107. Note that the processor 101, the memory 102, the storage device 103, the reading device 104, the communication interface 106, and the input/output interface 107 are mutually connected via, for example, a bus 108.

The processor 101 may be, for example, a single processor, a multiprocessor, or a multicore processor. The processor 101 reads and executes a program stored in storage device 103 and thereby operates as the acquisition portion 41, the calculation portion 42, and the output portion 46 described above. Note that the processor 101 is an example of an electric circuit.

The memory 102 is, for example, a semiconductor memory and may include a RAM region and a ROM region. The storage device 103 is, for example, a semiconductor memory such as a hard disk or a flash memory, or an external storage device.

The reading device 104, for example, accesses a removable storage medium 105 in accordance with an instruction of the processor 101. The removable storage medium 105 can be achieved by, for example, a semiconductor device, a medium at which information is inputted and outputted by magnetic action, and a medium at which information is inputted and outputted by optical action. Note that the semiconductor device is, for example, a USB (universal serial bus) memory. Further, the medium at which information is inputted and outputted by the magnetic action is, for example, a magnetic disk. The medium at which information is inputted and outputted by the optical action is, for example, a CD (compact disc)-ROM, a DVD (digital versatile disk), or a Blu-ray disc (Blu-ray is a registered trademark).

The communication interface 106 communicates with other devices, for example, in accordance with the instruction of the processor 101. The input/output interface 107 is an interface, for example, between an input/output device and the computer 100. The input device is, for example, a device which receives an instruction from the user such as a keyboard, a mouse, or a touch panel. The output device is, for example, a display device such as a display or a sound device such as a speaker.

The storage portion 47 described above may include, for example, the memory 102, the storage device 103, and the removable storage medium 105. Further, the acquisition portion 41 and the output portion 46 described above may include at least one of the input/output interface 107 and the communication interface 106.

The program executed by the processor 101 is provided to the computer 100, for example, in the following forms.

(1) Previously installed in the storage device 103

(2) Provided by the removable storage medium 105

(3) Provided from a server such as a program server

Note that the hardware configuration of the computer 100 for achieving the server device 40 described with reference to FIG. 18 is merely an example, and the embodiment is not limited thereto. For example, a part of the above configuration may be omitted, or a new configuration may be added to the above configuration. Further, in another embodiment, for example, a part or all of the functions of the calculation portion 42 described above may be implemented as hardware such as an FPGA (field programmable gate array), a SoC (system-on-a-chip), an ASIC (application specific integrated circuit), or a PLD (programmable logic device). That is, any electric circuit included in the server device 40 may perform the internal prediction processing described above.

The above-described embodiments illustrate specific examples in order to facilitate understanding of the invention, and the present invention is not limited to these embodiments. Variations obtained by modifying the above-described embodiments and alternatives to the above-described embodiments can be included. That is, in each embodiment, the constituents can be modified without departing from the spirit and scope thereof. In addition, a new embodiment can be implemented by appropriately combining a plurality of constituents disclosed in one or more embodiments. In addition, some constituents may be deleted from the constituents illustrated in the respective embodiments, or some components may be added to the constituents illustrated in the embodiments. Furthermore, the processing procedures described in each embodiment may be performed in a different order as long as there is no contradiction. That is, the internal prediction method of the cell aggregate, the program, the image processing device, and the system of the present invention can be variously modified and changed without departing from the scope of the invention defined by the claims.

The above embodiment describes the digital microscope including the digital camera 23 as an example. However, the imaging device for generating the image of the cell aggregate may be, for example, a laser scanning type microscope. Further, the imaging device for generating the image of the cell aggregate is not limited to the microscope, and other imaging devices may be used for generating the image of the cell aggregate.

The above embodiment describes the example where the internal structure of the cell aggregate is predicted by calculating the feature amount from each of the contours of the cell aggregates appeared in the tomographic images captured from the different directions and then quantifying the feature of the three-dimensional shape of the cell aggregate using the feature amount calculated from the two-dimensional image. However, the internal structure of the cell aggregate may be predicted by generating the three-dimensional image of the cell aggregates, calculating the feature amount from the three-dimensional image, and then quantifying the feature of the three-dimensional shape of the cell aggregate. Also, in this case, the feature amount desirably includes at least one of the first feature amount related to the unevenness on the surface of the cell aggregate and the second feature amount related to the deviation from the ideal shape of the cell aggregate, and more desirably, the feature amount includes both of them.

The above embodiment describes the three-dimensional model image and the tomographic image as an example of the structure information associated with the feature amount stored in the database. However, other pieces of information may be stored in the database. For example, information other than the image, such as the cell number (the live cell number, the dead cell number, etc.), a cell density, and the presence or absence, the size, and a ratio of voids present in the cell aggregate, may be included as the structure information. Further, the database may include, for example, information related to the quality of the cell aggregate, such as normal/abnormal and presence/absence of tumor, in addition to the structure information. Further, the database may include annotation information attached by the user.

The above embodiment describes an example where the information on the cell aggregate at a certain time point is stored in the database as the structure information of the cell aggregate. However, the database may include information related to a change over time in the cell aggregate which has been continuously observed. The server device 40 may determine whether a change in the cell aggregate specified by comparison of the prediction results performed at different timing is normal or abnormal by referring to the information related to the change over time in the database. For example, the server device 40 may determine whether the change is normal or abnormal on the basis of a proliferation rate, a proliferation number, or the like of the cells during a predetermined period of time.

In the above embodiment, the model image and the tomographic image are displayed as the internal prediction result. However, the image is not necessarily displayed, and other pieces of information may be displayed. For example, the number of the cells present in the cell aggregate, a cell proliferation rate or cell proliferation number during a predetermined period of time (e.g., a time period from the previous prediction to the present prediction), the quality information of the cell aggregate (normal/abnormal, the presence/absence of tumor, a survival rate (the live cell number/the total cell number)), and the like may be displayed.

The above embodiment describes an example where the server device 40 performs the internal prediction processing. However, the internal prediction processing may be performed in the microscope system 10 having captured the image of the cell aggregate. More specifically, the control device 30 may execute the internal prediction processing on the basis of the image generated by the microscope 20. Further, the internal prediction processing may be performed by a device different from the device in which the database is constructed. For example, the control device 30 may execute the internal prediction processing by referring to the database constructed in the server device 40.

As used herein, terms such as “first” and “second” that modify a noun do not limit the quantity or order of the elements represented by the noun. These terms are used only to distinguish between two or more elements. Therefore, the identification of the “first” and “second” elements does not mean that the “first” element precedes the “second” element. The identification of the “first” and “second” elements does not deny the existence of the “third” element.

What is claimed is:

1. An internal prediction method comprising:
acquiring an image of a cell aggregate;
calculating a feature amount related to a shape of the cell aggregate on the basis of the image; and
outputting structure information related to an internal structure of the cell aggregate on the basis of the feature amount.

2. The internal prediction method according to claim 1,
wherein acquiring the image of the cell aggregate includes acquiring two or more images obtained by imaging the cell aggregate from mutually different directions.

3. The internal prediction method according to claim 2,
wherein acquiring the two or more images includes acquiring an image obtained by imaging the cell aggregate from a first direction and an image obtained by imaging the cell aggregate from a second direction that intersects with the first direction.

4. The internal prediction method according to claim 3, wherein
acquiring the two or more images includes
acquiring a plurality of first images obtained by imaging mutually different surfaces of the cell aggregate from the first direction and
acquiring a plurality of second images obtained by imaging the mutually different surfaces of the cell aggregate from the second direction;
calculating the feature amount related to the shape of the cell aggregate includes calculating the feature amount on the basis of each of a third image selected from the plurality of the first images and a fourth image selected from the plurality of the second images; and
outputting the structure information related the internal structure includes acquiring the structure information on the basis of a positional relationship of the third image and the fourth image, the feature amount corresponding to the third image, and the feature amount corresponding to the fourth image.

5. The internal prediction method according to claim 4,
wherein calculating the feature amount related to the shape of the cell aggregate includes:
specifying a contour of the cell aggregate on the basis of each of the plurality of the first images and the plurality of the second images;
selecting the third image on the basis of a plurality of the contours corresponding to the plurality of the first images; and
selecting the fourth image on the basis of the plurality of the contours corresponding to the plurality of the second images.

6. The internal prediction method according to claim 1,
wherein calculating the feature amount related to the shape of the cell aggregate includes:
specifying a contour of the cell aggregate on the basis of the image;
displaying the contour on a display device;
receiving correction of the contour; and
calculating the feature amount on the basis of the corrected contour.

7. The internal prediction method according to claim 1,
wherein the feature amount includes a first feature amount related to unevenness on a surface of the cell aggregate.

8. The internal prediction method according to claim 2,
wherein the feature amount includes a first feature amount related to unevenness on a surface of the cell aggregate.

9. The internal prediction method according to claim 3,
wherein the feature amount includes a first feature amount related to unevenness on a surface of the cell aggregate.

10. The internal prediction method according to claim 7,
wherein calculating the feature amount related to the shape of the cell aggregate includes:
specifying a contour of the cell aggregate on the basis of the image;
calculating an approximate curve approximating the contour on the basis of the contour; and
calculating the first feature amount on the basis of the contour and the approximate curve.

11. The internal prediction method according to claim 1,
wherein the feature amount includes a second feature amount related to deviation from an ideal shape of the cell aggregate.

12. The internal prediction method according to claim 11,
wherein calculating the feature amount related to the shape of the cell aggregate includes:
specifying a contour of the cell aggregate on the basis of the image;
calculating a first radius of a circle circumscribing the contour and a second radius of a circle inscribed in the contour on the basis of the contour; and
calculating the second feature amount on the basis of the first radius and the second radius inscribed in the contour.

13. The internal prediction method according to claim 2,
wherein the feature amount includes a second feature amount related to deviation from an ideal shape of the cell aggregate.

14. The internal prediction method according to claim 1,
wherein the feature amount includes a first feature amount related to unevenness on a surface of the cell aggregate and a second feature amount related to deviation from an ideal shape of the cell aggregate.

15. The internal prediction method according to claim 1,
wherein outputting the structure information related to the internal structure includes outputting a model image representing a cell distribution in the cell aggregate predicted on the basis of the feature amount.

16. The internal prediction method according to claim 15,
wherein the model image includes a classification result obtained by classifying cells constituting the cell aggregate.

17. A non-transitory computer readable medium storing an internal prediction program of a cell aggregate,
wherein the program causes a computer to execute processes of:
acquiring an image of the cell aggregate;
calculating a feature amount related to a shape of the cell aggregate on the basis of the image; and
outputting structure information related to an internal structure of the cell aggregate on the basis of the feature amount.

18. An image processing device comprising:
an acquisition portion that acquires an image of a cell aggregate;
a calculation portion that calculates a feature amount related to a shape of the cell aggregate on the basis of the image; and
an output portion that outputs structure information related to an internal structure of the cell aggregate on the basis of the feature amount.

19. The image processing device according to claim 13, further
comprising a storage portion that stores the feature amount and the structure information in association with each other.

20. The image processing device according to claim 13, further
comprising a display portion that displays the structure information.
